(19) United States
(12) Patent Application Publication
(10) Pub. No.: US 2002/0016520 A1
(43) Pub. Date: Feb. 7, 2002

(54) Integrated process for the production of cumene

(75) Inventors: Alberto Paggini, Spino D'adda (IT); Domenico Sanfilippo, Paullo (IT); Elena Picciotto, Milan (IT)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: SNAMPROGETTI S.p.A., San Donato Milanese (IT)

(21) Appl. No.: 09/879,190

(22) Filed: Jun. 13, 2001

(30) Foreign Application Priority Data
Jun. 14, 2000 (IT) MI2000A 001326

(51) Int. Cl. C07C 2/64

(52) U.S. Cl. 585/323; 585/319

(57) ABSTRACT

Integrated process for the preparation of cumene which comprises dehydrogenating a stream of propane to propylene in a dehydrogenation unit and sending the stream leaving the dehydrogenation unit, containing 25-40% by weight of propylene, to an alkylation unit together with a stream of benzene with a molar ratio benzene/propylene ranging from 8 to 10. The alkylation product is distilled in a first distillation column to recover a light fraction, essentially consisting of propane which is recycled to the dehydrogenation, and a heavy fraction which is distilled in a second distillation column to recover non-reacted benzene at the head, recycled to the alkylation unit, and cumene with a purity of over 99%, at the tail.

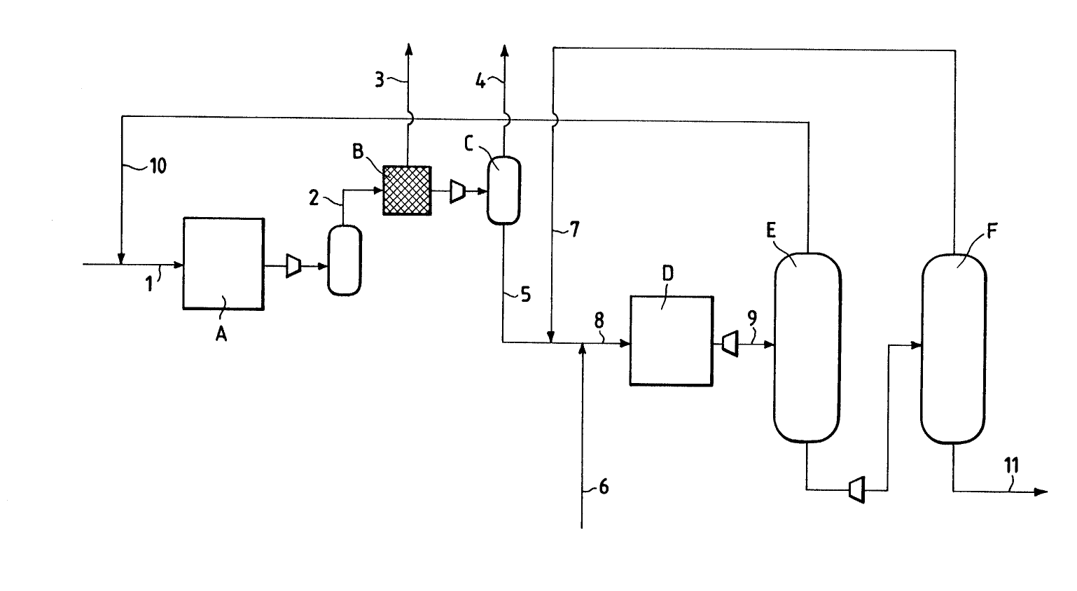

The present invention relates to an integrated process for the production of cumene.

More specifically, the present invention relates to a continuous process for the production of cumene by the alkylation of benzene with propylene, in which the dehydrogenation unit of propane to propylene and subsequent alkylation of benzene are integrated with each other.

The synthesis of cumene is known in literature. According to what is described by A. Chauvel, G. Lefebvre in “Petrochemical Processes”, Institut Francais du Petrol Publications, Editions Technip, 1989, pages 109-111, cumene can be prepared on an industrial scale starting from a stream containing propylene, chemical grade or refinery grade (92% purity), to which recycled propane is added in order to obtain a molar ratio propane/propylene of about 50/50. The propylene stream is fed together with benzene to a reactor containing a set of catalytic beds maintained at a temperature of 190-200° C. with injections of propane.

In order to avoid the formation of polyalkylated products, the reaction is carried out in the presence of a strong excess of benzene, the molar ratio benzene/propylene, in fact, is equal to 8-10, which is then recovered in a subsequent distillation phase.

U.S. Pat. No. 5,198,595 describes an alkylation process of aromatic compounds, in particular, the alkylation of benzene with propylene to give cumene, in which the problem of the formation of polyalkylated products is solved by the use of a catalyst selected from acid zeolites such as mordenite, in which the ratio silica/alumina is higher than 30.

More specifically, according to U.S. Pat. No. 5,198,595, cumene can be prepared by reacting a stream of propylene with a high degree of purity with benzene in molar ratios benzene/propylene of less than 8, as described in example 6. The catalyst is an acid mordenite with a symmetry index ranging from 0.5 to 1.3, calcined in air or in an inert atmosphere at a temperature ranging from 300 to 800° C. and subsequently treated with a strong acid such as nitric acid, hydrochloric acid and sulfuric acid.

Other methods for the preparation of cumene are described in U.S. Pat. No. 5,877,384 and German patent 3,437,615.

One of the main drawbacks of the known methods for preparing cumene is linked to the fact that the propylene used should have quite a high degree of purity. This necessity implies the use of a purification system, more or less forced, at the outlet of propylene production processes, whether they comprise the dehydrogenation of propane or its catalytic cracking. These purification systems inevitably influence the production costs of cumene.

The Applicant has, on the other hand, found that it is possible to prepare cumene by directly using the stream leaving the dehydrogenation unit of propane as an alkylating stream of benzene, without having to resort to intermediate fractionations.

The object of the present invention therefore relates to an integrated process for the preparation of cumene which comprises:

a. dehydrogenating a stream of propane to propylene in a dehydrogenation unit;

b. eliminating the hydrogen and possible reaction by-products from the stream leaving the dehydrogenation unit,

c. sending the stream leaving the dehydrogenation unit, containing 25-40% by weight of propylene, to an alkylation unit together with a stream of benzene with a molar ratio benzene/propylene ranging from 8 to 10;

d. distilling the alkylation product in a first distillation column to recover a light fraction, essentially consisting of propane which is recycled to the dehydrogenation unit, and a heavy fraction;

e. distilling the heavy fraction in a second distillation column to recover non-reacted benzene at the head, recycled to the alkylation unit, and cumene with a purity of over 99%, at the tail.

According to the present integrated process, the dehydrogenation unit operates at a temperature ranging from 450 to 800° C., at a pressure slightly higher than atmospheric pressure and in the presence of a dehydrogenation catalyst. Any dehydration catalyst of light paraffins can be used in the process object of the present invention, for example catalysts based on noble metals can be used, such as those described in U.S. Pat. Nos. 4,886,928 and 4,786,625, or catalysts based on chromium oxides, as described in English patent 2,162,082. Preferred catalysts, however, are a catalyst based on gallium oxide Ga₂O₃ and platinum supported on alumina, described in European patent 637,578, and a catalyst based on chromium oxide Cr₂O₃, tin oxide SnO and at least one oxide of an alkaline metal supported on alumina, described in European patent 894,781.

The stream leaving the dehydrogenation unit essentially consists of propylene, non-converted propane, hydrogen and other light products (methane). After a first compression phase at about 1-2 MPa, the stream is sent to a hydrogen separation unit operating with a membrane system and is then liquefied at about 2-5 MPa.

After elimination of the uncondensable products, the liquid stream is mixed with benzene and sent to the alkylation unit. The alkylation reaction is carried out with known methods, for example, according to the procedure described in European patent 432,814 with a zeolitic catalyst consisting, for example, of beta zeolite or ZSM-5, ZSM-12 zeolites or Y zeolite. The alkylation reaction is preferably carried out in liquid phase, at a temperature ranging from 110 to 150° C. and a pressure ranging from 2 to 5 MPa.

At the end of the alkylation, the reaction product, essentially consisting of propane, benzene and cumene, is expanded and subjected to a first distillation to recover propane at the head, which is recycled to the dehydrogenation unit, and a product at the tail, which, after further expansion, is subjected to a second distillation to separate the cumene produced from the benzene which in turn is recycled to the alkylation unit.

An illustrative but non-limiting example is provided for a better understanding of the present invention and for its embodiment.

### EXAMPLE

Reference is made to the scheme of the enclosed figure in which A represents a dehydrogenation unit of propane, B represents a membrane separation unit, C a container for collecting a liquid phase, D represents the alkylation reactor, E and F two distillation units.

A stream 1 is fed to the dehydrogenation reactor A, at a flow-rate of 4.4 Kg/h, consisting of 97% by weight of propane and 3% by weight of propylene. The dehydrogenation is carried out at a temperature of 585-590° C. and at a pressure of 0.124 MPa in the presence of the catalyst used in example 2 of European patent 637,578, with a conversion of propane equal to 35%.

The stream 2, leaving the dehydrogenation unit, consists of 71% by weight of propane, 27% of propylene and 2% of hydrogen and other by-products.

After compression at 2 MPa and cooling to room temperature, a membrane system B was used to separate the hydrogen from the dehydrogenated stream (stream 3 at 98% molar). On further compressing the stream leaving the membrane separator B to 4 MPa, a liquid phase is obtained, collected in C. The uncondensable products (CH₄) are discharged by means of 4.

4.357 Kg/h of liquid stream 5 (72% by weight of propane and 27% by weight of propylene and 1% of other by-products) are fed to the alkylation reactor D together with 2 Kg/h of fresh benzene (6) and 17.7 Kg/h of a stream (7) containing recycled benzene. The stream 8 thus obtained, consisting of 82% by weight of benzene, 13% of propane, 5% of propylene is fed to the alkylation reactor operating at a temperature of 120° C., a pressure of 4 MPa, with a ratio benzene/propylene of 9 and containing a catalyst consisting of beta zeolite. The propylene conversion was 92%.

The stream 9 leaving the alkylation reactor contains 12% by weight of cumene, 13% of propane, 73% of benzene and 2% of residual propylene and other by-products. After a first expansion to 1 MPa, the stream 9 is fed to the first distillation column E from which 3.3 Kg/h of a stream is recovered at the head, at 30° C. containing propane (95% by weight) and the residual propylene (3%), which are recycled by means of line 10 to the dehydrogenation unit. The product at the bottom, at a temperature of 180° C., after further expansion to 0.1 MPa, is fed to the second distillation column F from which recycled benzene (7) is recovered at the head, whereas 3 Kg of cumene (11) with a 99% purity, is recovered from the bottom.

1. An integrated process for the preparation of cumene which comprises:
a. dehydrogenating a stream of propane to propylene in a dehydrogenation unit;
b. eliminating the hydrogen and possible reaction byproducts from the stream leaving the dehydrogenation unit,
c. sending the stream leaving the dehydrogenation unit, containing 25-40% by weight of propylene, to an alkylation unit together with a stream of benzene with a molar ratio benzene/propylene ranging from 8 to 10;
d. distilling the alkylation product in a first distillation column to recover a light fraction, essentially consisting of propane which is recycled to the dehydrogenation unit, and a heavy fraction;
e. distilling the heavy fraction in a second distillation column to recover non-reacted benzene at the head, recycled to the alkylation unit, and cumene with a purity of over 99%, at the tail.

2. The process according to claim 1, wherein the dehydrogenation unit operates at a temperature ranging from 450 to 800° C.

3. The process according to claim 1 or 2, wherein the dehydrogenation catalyst is based on gallium oxide Ga₂O₃ and platinum supported on alumina.

4. The process according to claim 1 or 2, wherein the dehydrogenation catalyst is based on chromium oxide Cr₂O₃, tin oxide SnO and at least one alkaline metal oxide supported on alumina.

5. The process according to any of the previous claims, wherein the stream leaving the dehydrogenation unit is sent to a separation unit of the hydrogen operating with a membrane system.

6. The process according to any of the previous claims, wherein the alkylation reaction is carried out in the presence of a zeolitic catalyst, in liquid phase, at a temperature ranging from 110 to 150° C. and a pressure ranging from 2 to 5 MPa.
